(19) United States
(12) Patent Application Publication
(10) Pub. No.: US 2019/0125908 A1
(43) Pub. Date: May 2, 2019

(54) SYSTEM AND METHOD FOR IMPROVED CLEANING AND SANITIZING OF SURFACES USING ELECTROLYZED SOLUTIONS

(71) Applicant: Spraying Systems Co., Glendale Heights, IL (US)

(72) Inventors: James Barry Swartz, Noblesville, IL (US); Laura Jane Louis, Boise, ID (US); Julie Kay Thorpe, Noblesville, IN (US); James I. Moyer, Kokomo, IN (US); Kevin Patrick Shane, Kokomo, IN (US); Matthew Auburn Peterson, Zionsville, IN (US)

(21) Appl. No.: 16/179,203

(22) Filed: Nov. 2, 2018

Related U.S. Application Data
(60) Provisional application No. 62/580,800, filed on Nov. 2, 2017.

(51) Int. Cl. A61L 2/18; B05B 9/08; B05B 1/04

(52) U.S. Cl. CPC A61L 2/18; B08B 3/026; B05B 1/04; B05B 9/0861

(57) ABSTRACT

A system for cleaning and sanitizing a surface using electrolyzed solutions is provided including a first container holding an electrolyzed base cleaner and a second container holding an electrolyzed acid sanitizer. A spray device is in communication with the first container and with the second container. The spray device has a spray nozzle and an actuator for effecting discharge of the electrolyzed base cleaner and the electrolyzed acid sanitizer via the spray nozzle. A controller is in communication with the actuator and configured to successively and alternately direct the discharge of the base cleaner and the acid sanitizer from the spray nozzle of the spray device upon actuation of the actuator.

### CROSS-REFERENCE TO RELATED APPLICATIONS

This patent application claims the benefit of U.S. Provisional Patent Application No. 62/580,800, filed Nov. 2, 2017, which is incorporated herein by reference.

### BACKGROUND OF THE INVENTION

Cleaning and sanitizing surfaces on a regular basis can help prevent the spread of infectious diseases. Fats and proteins on surfaces can shelter pathogens and provide them with a source of food. Surfaces cannot be effectively rid of pathogens without removing these fats and proteins. Accordingly, most cleaning and sanitizing processes are two steps, first removing and/or denaturing of fats and proteins through cleaning and then sanitizing. Each of these steps requires spraying and then wiping, which is time consuming and leads to increased labor costs. Chemicals that can both clean and disinfect in a single step are often toxic and expensive to produce and maintain.

Systems and methods are known that electrolyze water containing alkali salts to produce acidic electrolyzed water and alkaline electrolyzed water. Acidic electrolyzed water, which typically has a pH between about 5.0 and about 6.0, generally comprises a disinfectant that is increasingly used in a variety of sanitizing applications including in the medical, agricultural and food processing industries and in other institutional environments. The alkaline (i.e., basic) electrolyzed water, which typically has a pH between about 12.0 and 12.5, also has a sanitizing as well as a detergent and denaturing effect and is useful in cleaning a variety of stains, including oil and grease stains. As with conventional cleaning solution, acidic electrolyzed water and alkaline electrolyzed water must be applied in separate steps when cleaning and sanitizing a surface. When the two solutions are mixed together so that they can be applied in a single step, the resultant solution typically has a pH of about 10.0 to about 10.5 which greatly reduces its ability to saponify or denature fats and proteins and kill pathogens. Thus, the resultant mixed solution is less effective as both a cleaner and a disinfectant.

### BRIEF SUMMARY OF THE INVENTION

Accordingly, in view of the foregoing, a general object of the present invention is to provide an improved system and method for cleaning and sanitizing a surface that can be performed using a single spray and single wipe thus allowing the cleaning and sanitizing operation to be performed more quickly and easily than existing cleaning and sanitizing systems.

A related object of the present invention is to provide a system and method for cleaning and sanitizing a surface of the foregoing type than reduces labor costs.

A further object of present invention is to provide a system and method for cleaning and sanitizing a surface that is easier to operate and perform.

Another object of the present invention is to provide a system and method for cleaning and sanitizing a surface of the foregoing type that is at least as effective as two spray/two wipe systems.

Another object of the present invention is to provide a system and method for cleaning and sanitizing a surface that helps reduce repetitive motion injuries associated with cleaning and sanitizing operations.

A further object of present invention is to provide a system and method for cleaning and sanitizing a surface that allows for use of safer and lower cost chemicals.

A further object of the present invention is to provide a system and method for cleaning and sanitizing a surface that is usable on a variety of surfaces.

Other objects and advantages of the present invention will become apparent upon reading the following detailed description and upon reference to the drawings.

### BRIEF DESCRIPTION OF THE SEVERAL VIEWS OF THE DRAWINGS

FIG. 1 is a partially exploded perspective view of an exemplary single spray/single wipe cleaning and sanitizing system in accordance with the present invention.

FIG. 2 is a partial, cutaway view of the solution feed lines and control leads for the cleaning and sanitizing system of FIG. 1.

FIG. 3A is a perspective view of one embodiment of a spray device of the cleaning and sanitizing system of FIG. 1.

FIG. 3B is an end view of one embodiment of a spray nozzle of the spray device of FIG. 3A.

FIG. 3C is a side view of the spray nozzle of FIG. 3B.

FIG. 4A is a perspective view of an alternative embodiment of a spray device of the cleaning and sanitizing system of FIG. 1.

FIG. 4B is an end view of one embodiment of a spray nozzle of the spray device of FIG. 4A.

FIG. 4C is a side view of the spray nozzle of FIG. 4B.

FIG. 5 is a partially exploded perspective view of an alternative embodiment of a single spray/single wipe cleaning and sanitizing system according to the present invention.

FIG. 6 is a diagrammatic top view of a series of discharges of cleaner and sanitizer according to one embodiment of the present invention.

### DETAILED DESCRIPTION OF THE INVENTION

The present invention relates to a system and method for cleaning and sanitizing a surface that only requires a single spray and single wipe to both clean and sanitize the surface. The system and method may use electrolyzed solutions including, for example, an alkaline electrolyzed water solution as the cleaning solution and an acidic electrolyzed water solution as the sanitizing solution. Advantageously, the system and method minimizes mixing of the cleaner and sanitizer, which helps maximize effectiveness of the individual solutions. Additionally, as discussed further below, the capability of automatically discharging both a cleaner and a sanitizer with a single actuation of the system makes the system much easier for an operator to use. Thus, the system and method of the present invention can be operated and/or performed considerably more quickly than existing cleaning systems which can result in substantially lower labor costs in commercial cleaning operations.

The cleaning and sanitizing of the present invention uses electrochemically activated solutions including, for example, electrolyzed water solutions. In particular, the electrolyzed water solutions may be the result of a process in which a solution of water and an alkali salt is electrolyzed to produce acidic electrolyzed water and/or alkaline (i.e., base) electrolyzed water. Both acidic electrolyzed water (i.e., acid sanitizer) and alkaline electrolyzed water (i.e., base cleaner) have beneficial sanitizing and cleansing properties making them useful in a variety of applications including medical, agricultural, food processing and institutional. According to one embodiment, the water and salt solution is a saline or brine solution comprising water and sodium chloride. Depending on the process conditions, electrolysis of a brine solution comprising water and sodium chloride produces aqueous hypochlorous acid solution (e.g., an acid sanitizer) and aqueous sodium hydroxide solution (e.g., a base cleaner), each being an aqueous chemical solution. As will be appreciated by those skilled in the art, the present disclosure is not limited to electrolysis of any particular solution or use in any particular application.

To provide improved cleaning and sanitizing of a surface, the cleaning and sanitizing system of the present invention is operable to separately store and then sequentially discharge the electrolyzed base cleaner and electrolyzed acid sanitizer upon a single actuation of the system. To this end, the system may include a single spraying device with a spray nozzle that is configured such that the spray nozzle is automatically fed alternately with the electrolyzed base cleaner and then the electrolyzed acid sanitizer upon triggering of an actuator on the spraying device. The system may be configured such that the time gap between the discharge of the electrolyzed base cleaner and the electrolyzed acid sanitizer may be electronically adjustable.

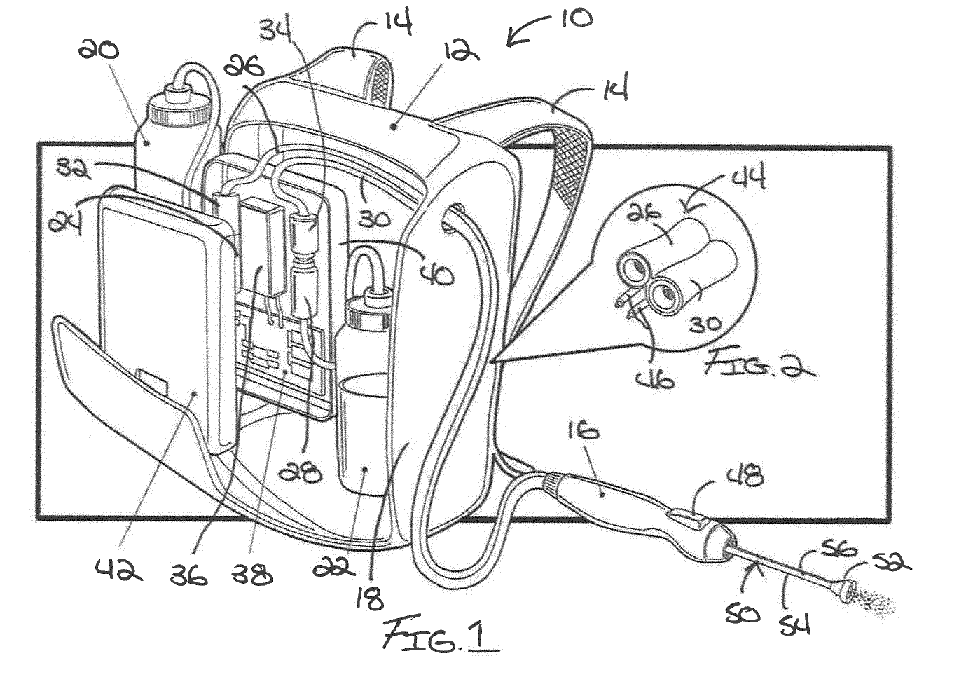

Referring to FIG. 1 of the drawings, an exemplary embodiment of a cleaning and sanitizing system 10 according to the present invention is shown. The illustrated cleaning and sanitizing system 10 is configured as a backpack 12 with attached shoulder straps 14 although it should be understood that other configurations may also be used. The cleaning and sanitizing system 10 of FIG. 1 includes a spray device 16 that is operatively connected to a base unit 18 that can be carried on the back of an operator. The interior of the base unit 18 can be configured to house an electrolyzed base cleaner container 20 and an electrolyzed acid sanitizer container 22. In this case, the cleaner container 20 and the sanitizer container 22 are arranged on opposite sides of the interior of the base unit 18.

To drive movement of the electrolyzed solutions from the base unit 18 to the spray device 16, a first cleaner feed pump 24 is provided in the base unit 18 that is arranged and configured to draw cleaner out of the cleaner container 20 and direct it through a cleaner feed line 26. Additionally, a second sanitizer feed pump 28 is provided in the base unit 18 that is arranged and configured to draw sanitizer out of the sanitizer container 22 and direct it through a sanitizer feed line 30. The cleaner and sanitizer feed pumps 24, 28 may each be driven by an associated electric motor 32, 34 that are powered by a power source 36 that is carried in the base unit 18. The power source 36 may be, for example, a rechargeable battery. The base unit 18 may further include a controller 38 that is configured to direct operation of the pumps 24, 28 and the spray device 16 as further described below. The pumps 24, 28, power source 36 and controller 38 may be arranged together in a casing 40 having a removable cover 42 that can be arranged between the cleaner container 20 and the sanitizer container 22 as shown in FIG. 1.

For directing the electrolyzed base cleaner and electrolyzed acidic sanitizer to the spray device 16, the cleaner feed line 26 and the sanitizer feed line 30 extend from the base unit 18 to the spray device 16. More specifically, in the illustrated embodiment, the cleaner and sanitizer feed lines 26, 30 are secured together to form a single cord 44 attaching the spray device 16 to the base unit 18 as shown, for example, in FIG. 2. In addition to the feed lines 26, 30, the cord 44 to the spray device 16 may also include attached control leads 46 that extend from the controller 38 in the base unit 18 to the spray device 16 in order to direct control signals between the controller and the spray device. In an alternative embodiment, at least a portion of the cleaner feed line 262 and the sanitizer feed line 30 between the base unit 18 and the spray device 16 may be configured as a single tube that is divided into two separate fluid passages one of which carries the cleaner and one of which carries the sanitizer.

In the illustrated embodiment, the spray device 16 is configured as a hand-operated spray wand with an actuator, in this case, an actuator switch 48 arranged on a side thereof. The wand-like configuration of the spray device 16 helps the spray device to be easily gripped and aimed by an operator. The actuator switch 48 may be in communication with the controller 38, such as through the control leads 46. Moreover, the controller 38 may be configured such that upon actuation of the actuator switch 48 on the spray device 16 the controller 38 successively directs first the cleaner feed pump 24 to direct base cleaner from the cleaner container 20 to the spray device 16 and then directs the sanitizer feed pump 28 to direct acidic sanitizer from the sanitizer container 22 to the spray device. According to one embodiment, the actuator 48 may be configured as an electronic trigger which along with the controller 38 is configured such that once the actuator 48 is depressed once the spray device 16 will alternately spray cleaner and sanitizer as long as the actuator is depressed. Thus, spray is discharged automatically and in a continuous alternating sequence of cleaner and sanitizer (or vice versa) upon a single actuation of the actuator 48. This can significantly reduce the risk of repetitive motion injuries caused by repetitive squeezing of a trigger of, for example, a spray bottle.

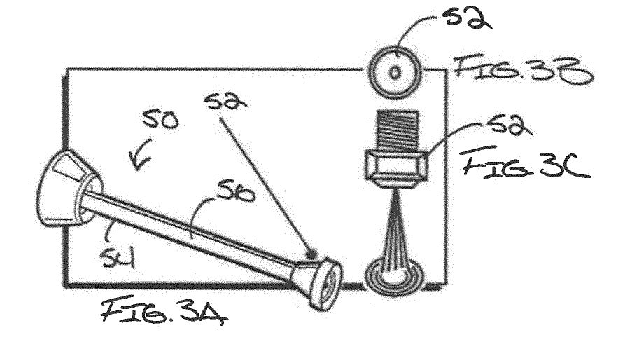

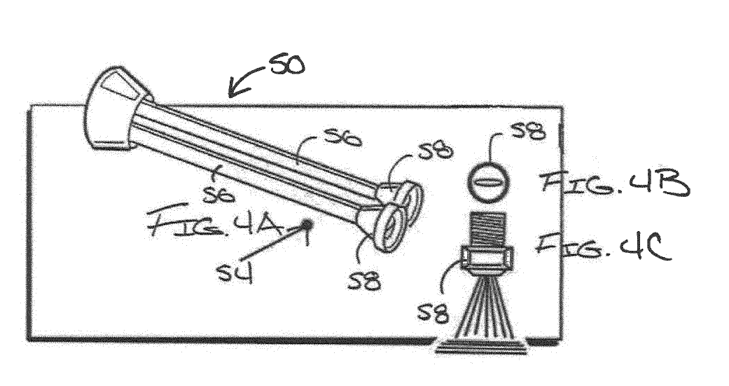

For shaping and directing the discharging electrolyzed base cleaner and the electrolyzed acid sanitizer, the spray device 16 may be equipped with one or more removable and replaceable spray nozzles 50. In FIGS. 3A-C, a single spray nozzle 50 is shown which has a spray tip 58 configured to produce a cone-shaped discharge pattern. The single spray nozzle 50 has an elongated body 54 that defines, in this case, a single fluid passage 56 for directing fluid introduced into the spray device 16 to the spray tip 58. This nozzle body 54 is removably attached to the spray device 16 such as shown in FIG. 1. Upon actuation of the actuator switch 48 on the spray device 16, the system 10 may be configured to sequentially discharge first cleaner and then sanitizer through the single spray tip 58. While the illustrated embodiment uses a cone-shaped discharge pattern (see, e.g. FIG. 3C), the spray tip 58 may be configured to produce any desired discharge pattern.

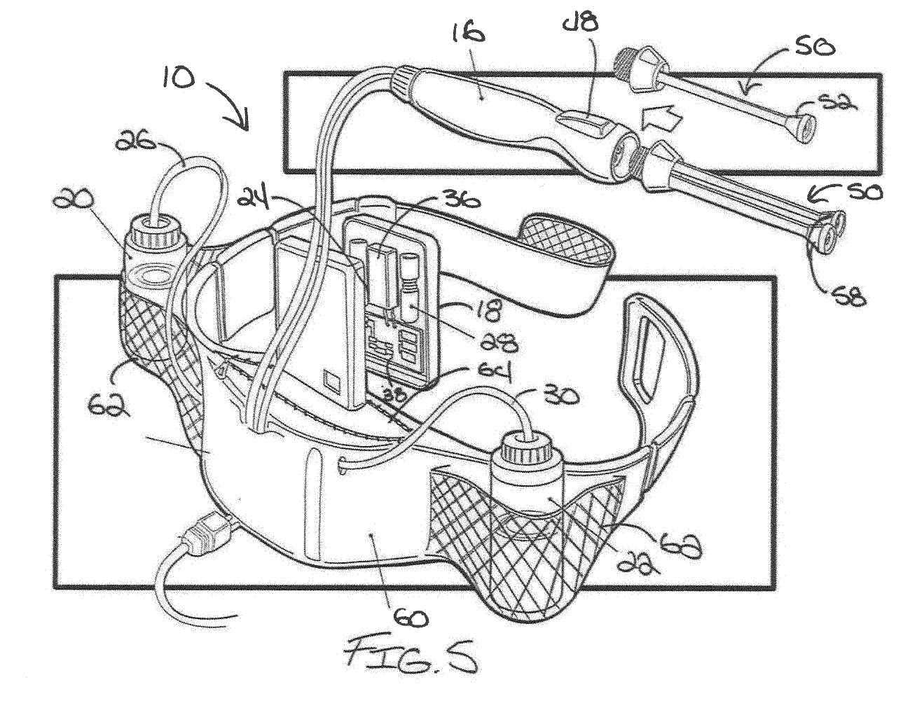

A spray nozzle 50 with a dual configuration that also may be used with the spray device 16 of FIG. 1 is shown in FIGS. 4A-C. As in the embodiment of FIGS. 3A-C, the spray nozzle 50 of FIGS. 4A-C includes an elongated body 54, however in this case the body comprises two separate fluid passages 56, one for directing cleaner and one for directing sanitizer. Each fluid passage 56 of the nozzle body 54 terminates in a separate spray tip 58, which in the illustrated embodiment is configured to produce a flat fan-shaped discharge pattern. The system 10 may be configured such that upon actuation of the switch 48 on the spray device 16, the system will first spray cleaner out of one spray tip 58 and then sanitizer out of the other spray tip 58 in a side-by-side arrangement. While examples of possible spray nozzle 50 and spray tip 52, 58 configurations are shown, it should be appreciated that different nozzle and tip arrangements could also be used. In particular, the nozzles can be configured to produce different effects as desired.

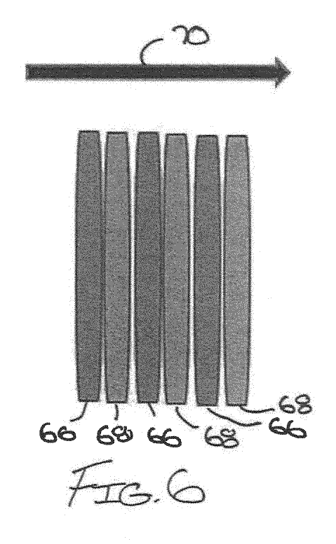

An alternative embodiment of a cleaning and sanitizing system 10 according to the present invention is shown in FIG. 5. In the embodiment of FIG. 5, the cleaning and sanitizing system 10 is configured as a waist belt-supported unit that is otherwise very similarly configured to the backpack unit of FIG. 1. The system 10 of FIG. 5 includes a belt carrier 60 having two external side pockets 62, one to support the electrolyzed base cleaner container 20 and one to support the electrolyzed acid sanitizer container 22. In the illustrated embodiment, the base unit 18 containing the controller 38, pumps 24, 28 and power source 36 is supported in a central pocket 64 defined by the belt carrier 60. Like in the embodiment of FIG. 1, the spray device 16 of the FIG. 5 embodiment is configured to receive interchangeable spray nozzles 50 and tips 52 and is connected to the base unit 18 via the cleaner and sanitizer feed lines 24, 30. While backpack and belt type units are shown in the drawings, the present invention is not intended to be limited to systems having such configurations. Rather, the present invention is intended to cover any system configuration capable of separately storing electrolyzed base cleaner and electrolyzed acid sanitizer and then separately discharging upon a single actuation of the system the base cleaner and the acid sanitizer.

In use, according to one embodiment, an operator moves the spray device 16 across a surface to be cleaned and sanitized while triggering the actuator switch 48 on the spray device 16. Upon actuation of the switch 48, the electrolyzed cleaner and the electrolyzed sanitizer are discharged so as to produce side-by-side discharge patterns of the cleaner and sanitizer. This process can be repeated as the operator moves the spray device 16 across the surface. This results in substantially separate, but adjacent, discharge patterns of the electrolyzed base cleaner and the electrolyzed acid sanitizer. One example of the resulting discharge patterns of the cleaner and sanitizer are shown in FIG. 6. In FIG. 6, an array of alternating flat fan-shaped discharge patterns of base cleaner (referenced as 66) and acid sanitizer (referenced as 68) are shown. Once the base cleaner and acid sanitizer have been laid down on the surface to be cleaned, the user can then wipe (in a single wiping action) the discharged cleaner and sanitizer in the same direction (referenced in FIG. 6 by the arrow 70) that the spray device 16 was moved across the surface to be cleaned. Depending upon the number and/or type of spray nozzles 50 provided on the spraying device 16, the cleaner and sanitizer may be discharged sequentially, such as when a single spray nozzle 50 is used, or simultaneously, such as when two or more spray nozzles 50 are provided. As noted above, the actuator switch 48 on the spray device 16 and the controller 38 may be configured such that a single press of the switch produces one dual burst of cleaner and sanitizer or the switch 48 may be held down for repeated bursts of cleaner and sanitizer until the switch is released.

With embodiments of the invention producing simultaneous discharge of the cleaner and sanitizer (e.g., the embodiment of FIGS. 4A-C), it may be advantageous to configure the spray device such that both the cleaner and sanitizer have a droplet size of greater than approximately 100 microns. In such a case, the liquid nozzle pressure can be adjusted and controlled such that most of the disinfectant will not mix with the cleaner. Thus, the system can produce independent, yet simultaneous, discharges of cleaner and disinfectant that can then be wiped in a single wipe, allowing a surface to be both cleaned and disinfected in a single step.

As compared with existing cleaning and sanitizing processes that utilize separate cleaners and sanitizer, the process and system of the present invention allows the cleaning and sanitizing process to be performed more quickly and with less repetitive motion stress on the user because of the ability to discharge both the cleaner and sanitizer with a single actuation of the system. The dual dispensing of the cleaner and sanitizer also allows both to be captured in a single wiping action, which again saves time during the cleaning and sanitizing process. These time savings can help reduce labor costs in institutional or commercial cleaning and sanitizing operations. As compared to existing cleaning and sanitizing systems and processes that use a cleaner and sanitizer mixture, the system and process of the present invention allows for the use of lower cost and safer electrolyzed cleaning and sanitizing solutions that are useable on a wide variety of different surfaces. Additionally, the electrolyzed cleaning and sanitizing solutions allows for the use of reusable containers to store the cleaner and sanitizer, which again can result in a cost savings.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A system for cleaning and sanitizing a surface using electrolyzed solutions comprising:
a first container holding an electrolyzed base cleaner;
a second container holding an electrolyzed acid sanitizer;
a spray device in communication with the first container via a cleaner feed line and with the second container via a sanitizer feed line, the spray device having a spray nozzle and having an actuator for effecting discharge of the electrolyzed base cleaner and the electrolyzed acid sanitizer via the spray nozzle; and
a controller in communication with the actuator and configured to successively and alternately direct the discharge of the base cleaner and the acid sanitizer from the spray nozzle of the spray device upon actuation of the actuator.

2. The system for cleaning and sanitizing a surface of claim 1 wherein the controller is further configured to successively and alternately direct the discharge of first the base cleaner and then the acid sanitizer from the spray device continuously while the actuator is actuated.

3. The system for cleaning and sanitizing a surface of claim 1 wherein the spray nozzle has a single spray tip through which both the base cleaner and the acid sanitizer are discharged.

4. The system for cleaning and sanitizing a surface of claim 1 wherein the spray device is configured as a spray wand.

5. The system for cleaning and sanitizing a surface of claim 1 further including a cleaner feed pump arranged configured to draw cleaner out of the first container and direct the cleaner through the cleaner feed line and a sanitizer feed pump arranged and configured to draw sanitizer out of the second container and direct the sanitizer through the sanitizer feed line, wherein the controller communicates with and directs operation of the cleaner feed pump and the sanitizer feed pump.

6. The system for cleaning and sanitizing a surface of claim 3 wherein the spray tip is configured to produce a fan-shaped discharge pattern.

7. The system for cleaning and sanitizing a surface of claim 1 wherein the first container and the second container are supported on a backpack.

8. The system for cleaning and sanitizing a surface of claim 1 wherein the first container and the second container are supported on a belt.

9. A system for cleaning and sanitizing a surface using electrolyzed solutions comprising:
a first container holding an electrolyzed base cleaner;
a second container holding an electrolyzed acid sanitizer;
a spray device in communication with the first container via a cleaner feed line and with the second container via a sanitizer feed line, the spray device having a spray nozzle with a first spray tip for discharging the base cleaner and a second spray tip for discharging the acid sanitizer, the first and second spray tips being configured to produce side-by-side discharge patterns, the spray device having an actuator for effecting discharge of the base cleaner from the first spray tip and the acid sanitizer via the second spray tip; and
a controller in communication with the actuator and configured to direct the discharge of the base cleaner and the acid sanitizer from the spray nozzle of the spray device upon actuation of the actuator.

10. The system for cleaning and sanitizing a surface of claim 9 wherein the controller is further configured to successively and alternately direct the discharge of first the base cleaner and then the acid sanitizer upon actuation of the actuator.

11. The system for cleaning and sanitizing a surface of claim 10 wherein the controller is further configured to successively and alternately direct the discharge of first the base cleaner and then the acid sanitizer from the spray device continuously while the actuator is actuated.

12. The system for cleaning and sanitizing a surface of claim 9 wherein the spray device is configured as a spray wand.

13. The system for cleaning and sanitizing a surface of claim 9 further including a cleaner feed pump arranged configured to draw cleaner out of the first container and direct the cleaner through the cleaner feed line and a sanitizer feed pump arranged and configured to draw sanitizer out of the second container and direct the sanitizer through the sanitizer feed line, wherein the controller communicates with and directs operation of the cleaner feed pump and the sanitizer feed pump.

14. The system for cleaning and sanitizing a surface of claim 9 wherein the spray tip is configured to produce a fan-shaped discharge pattern.

15. The system for cleaning and sanitizing a surface of claim 9 wherein the first container and the second container are supported on a backpack.

16. The system for cleaning and sanitizing a surface of claim 9 wherein the first container and the second container are supported on a belt.

17. A method of cleaning and sanitizing a surface comprising the steps of:
moving a spray device across the surface;
successively and alternately discharging first an electrolyzed base cleaner and then an electrolyzed acid sanitizer from the spray device as the spray device is moved across the surface in a first direction so as to produce side-by-side discharge patterns of the base cleaner and the acid sanitizer; and
wiping the discharged base cleaner and acid sanitizer in the first direction.

18. The method of claim 17 wherein the discharge patterns are fan-shaped.

19. The method of claim 17 wherein actuating an actuator on the spray device discharges the base cleaner and the acid sanitizer.

20. The method of claim 19 wherein the actuating the actuator produces a continuous, successive and alternating discharge of the base cleaner and the acid sanitizer while the actuator is actuated.
